(19) United States
(12) Patent Application Publication
(10) Pub. No.: US 2009/0093868 A1
(43) Pub. Date: Apr. 9, 2009

(54) Laser Apparatus for Medical Treatment Using Pulse Trains

(76) Inventors: Guang Hoon Kim, Millak-dong (KR); Dae Sic Lee, Ansan-si (KR); Uk Kang, Gunpo-si (KR); Geun Hie Rim, Changwon-si (KR); R. Andreev, Saint Petersburg (RU); A. Bogoyavlensky, Saint Petersburg (RU); S. Khankov, Saint Petersburg (RU)

Correspondence Address: FROMMER LAWRENCE & HAUG LLP, 745 FIFTH AVENUE, NEW YORK, NY, 10151, US

(21) Appl. No.: 12/169,322

(22) Filed: Jul. 8, 2008

(30) Foreign Application Priority Data
Oct. 9, 2007 (KR) 10-2007-0101188

(51) Int. Cl. A61B 18/20

(52) U.S. Cl. 607/89

(57) ABSTRACT

The present invention provides a laser apparatus for medical treatment using pulse trains, in which the skin is cooled to a given temperature and then one or more laser pulses are applied to a to-be-treated region at specific intervals, thereby alleviating a pain associated with a skin disease and activating a dermis layer. According to the present invention, a pain caused during the treatment process is alleviated by using laser pulse trains and a cooling unit, and heat energy is transferred to only a dermis layer of a to-be-treated region without eliminating an epidermis layer of the to-be-treated region, thereby remarkably improving the treatment recovery period and the frequency of treatments as compared to the conventional treatment method.

### CROSS-REFERENCE TO RELATED APPLICATION

This application claims the benefit under 35 U.S.C. §119(a) of Korean Patent Application No.10-2007-0101188 filed on Oct. 9, 2007, the entire contents of which are incorporated herein by reference.

### BACKGROUND OF THE INVENTION

(a) Field of the Invention

The present invention relates to a laser apparatus for medical treatment using pulse trains. More particularly, the present invention relates to a laser apparatus for medical treatment using pulse trains, in which the skin is cooled to a given temperature and then one or more laser pulses are applied to a region to be treated (also called ‘to-be-treated region’) at specific intervals, thereby alleviating a pain associated with a skin disease and activating a dermis layer.

(b) Background Art

In order to improve a long-term recovery period and a sequelae, a skin treatment method using a laser is currently spotlighted in which several hundreds to hundred thousands of micro treatment zones are formed per 1 cm² and medical treatment is given to each of the micro treatment zones using a laser.

A laser beam from a laser is irradiated onto a plurality of laser spots formed on a to-be-treated object so as to irradiate the laser beam onto the several hundreds to hundred thousands of micro treatment zones. Such laser spots are formed using a conventional laser scanner.

A skin treatment method using the laser is performed in such a fashion that micro treatment zones are formed on a to-be-treated region and energy is transferred to a given depth of the micro treatment zones through a microlens array to thereby treat the damaged skin.

However, such a conventional skin treatment method using the laser is a method in which the micro treatment zones are formed on a to-be-treated region so as to partially cure only approximately 10 to 30% of a whole treatment region to thereby remarkably improve sequelae caused after treatment as compared to a whole laser treatment.

However, in the whole laser treatment method and the partial micro treatment method, a pain caused during the medical treatment is the greatest shortcoming.

Furthermore, since the partial micro treatment method partially cures only approximately 10 to 30% of the whole treatment region, a treatment efficiency is deteriorated, resulting in performing several medical procedures.

The information disclosed in this Background of the Invention section is only for enhancement of understanding of the background of the invention and should not be taken as an acknowledgment or any form of suggestion that this information forms the prior art that is already known to a person skilled in that art.

### SUMMARY OF THE INVENTION

The present invention has been made in an effort to solve the above problems occurring in the prior art, and it is an object of the present invention to provide a laser apparatus for medical treatment using pulse trains, in which a pain caused during the treatment process is alleviated by using laser pulse trains and a cooling unit, and heat energy is transferred to only a dermis layer of a to-be-treated region without eliminating an epidermis layer of the to-be-treated region, thereby remarkably improving the treatment recovery period and the frequency of treatments as compared to the conventional treatment method.

In order to accomplish the above object, according to a preferred embodiment of the present invention, there is provided a laser apparatus for medical treatment using pulse trains, comprising:

a laser pulse generator for generating a plurality of laser pulses;

an optical energy transfer element connected at one end thereof to the laser pulse generator and for transferring optical energy of the laser pulses generated from the laser pulse generator;

a handpiece connected at one end thereof to the other end of the optical energy transfer element for irradiating the optical energy of the laser pulses transferred thereto from the laser pulse generator by the optical energy transfer element to a to-be-treated region while being close contact with the to-be-treated region;

a cooling unit mounted at a distal end of the handpiece for cooling the skin of the to-be-treated region; and

a controller for adjusting the width of and the optical energy of the laser pulses generated from the laser pulse generator in response to an external input signal, and controlling a skin cooling temperature through the cooling unit,

whereby the skin of the to-be-treated region is cooled to a given temperature by the cooling unit, and then the plurality of laser pulses generated from the laser pulse generator are irradiated to the to-be-treated region at specific intervals.

In a preferred embodiment, the wavelengths of the laser pulses generated from the laser pulse generator ranges from 1400 nm to 1500 nm.

In a more preferred embodiment, the pulse width of each of the laser pulses generated from the laser pulse generator ranges from 50 ms to 300 ms.

Also, in a preferred embodiment, the interval between the laser pulses generated from the laser pulse generator ranges from 50 ms to 300 ms.

In addition, in a preferred embodiment, the diameter of a spot formed by each of the laser pulses irradiated to the to-be-treated region through the handpiece ranges from 5 mm to 15 mm.

Further, in a preferred embodiment, the cooling temperature of the skin cooled through the cooling unit ranges from 3° C. to 10° C.

### BRIEF DESCRIPTION OF THE DRAWINGS

FIG. 1 is a graph showing a variation in temperature at the epidermis and the dermis when the optical energy of the laser pulses is irradiated to the skin for 200 ms without cooling the skin;

FIG. 2 is a graph showing a relationship between the skin depth and the temperature at the epidermis when the trains of the laser pulses are incident on the epidermis with a depth of 50 μm after cooling the skin according to an embodiment of the present invention;

FIG. 3 is a graph showing a relationship between the skin depth and the temperature at the epidermis when the trains of the laser pulses are incident on the epidermis with a depth of 100 μm after cooling the skin according to an embodiment of the present invention;

FIG. 4 is a graph showing the relationship between the skin depth and the temperature at the epidermis when the trains of the laser pulses are incident on the epidermis with a depth of 150 μm after cooling the skin according to an embodiment of the present invention;

FIG. 5 is a graph showing the relationship between the magnitude of optical energy and the interval between laser pulses according to an embodiment of the present invention;

FIG. 6 is a diagrammatic view showing a degree in which heat is dispersed in the skin by the pulse trains;

FIG. 7 is a cross-sectional view showing the structure of a general skin;

FIG. 8 is a graph showing an absorption coefficient and a scattering coefficient by wavelength in an epidermis layer and a dermis layer of the skin; and

FIG. 9 is a diagrammatic view showing the construction of a laser apparatus for medical treatment using pulse trains according to an embodiment of the present invention.

Reference numerals set forth in the Drawings includes reference to the following elements as further discussed below:

10: epidermis

11: dermis

12: horny layer

13: granular layer

14: spinous layer

15: basal cell layer

16: laser pulse generator

17: optical energy transfer element

18: handpiece

19: cooling unit

### DETAILED DESCRIPTION

Reference will now be made in detail to the preferred embodiment of the present invention, examples of which are illustrated in the drawings attached hereinafter, wherein like reference numerals refer to like elements throughout. The embodiments are described below so as to explain the present invention by referring to the figures.

Now, a preferred embodiment of according to the present invention will be described hereinafter in detail with reference to the accompanying drawings.

The main tissue inside the skin is formed of a gelatinous tissue of a skin layer, and the ratio of collagen (protein=fiber) occupying the inside of the skin is approximately 90%. A colloidal element is fully filled between fibers.

As one grows older, the activity of the skin tissue is gradually degenerated and the amount of collagen is reduced and hardened. Also, the moisture retention capacity of the skin tissue is degraded and the skin loses elasticity. The skin tissue is even ruptured.

As a result, the skin is susceptible to irritation and damage, and becomes dry so that black speckles and fine wrinkles are caused on the surface thereof.

Collagen is a protein which is contained in a large amount in each region of the human body and serves as a ring that interconnects cells. Collagen also serves to maintain gloss and elasticity of the skin.

Collagen occupies one third of the total protein constituting the human body and plays an important role in promotion of blood circulation of our whole body.

Collagen interconnects a cell and a cell and nutrients absorbed in the small intestine are transferred to each cell by means of collagen. In addition, a metabolic waste product is carried to a bloody vessel through collagen so as to be discharged to the outside of the human body.

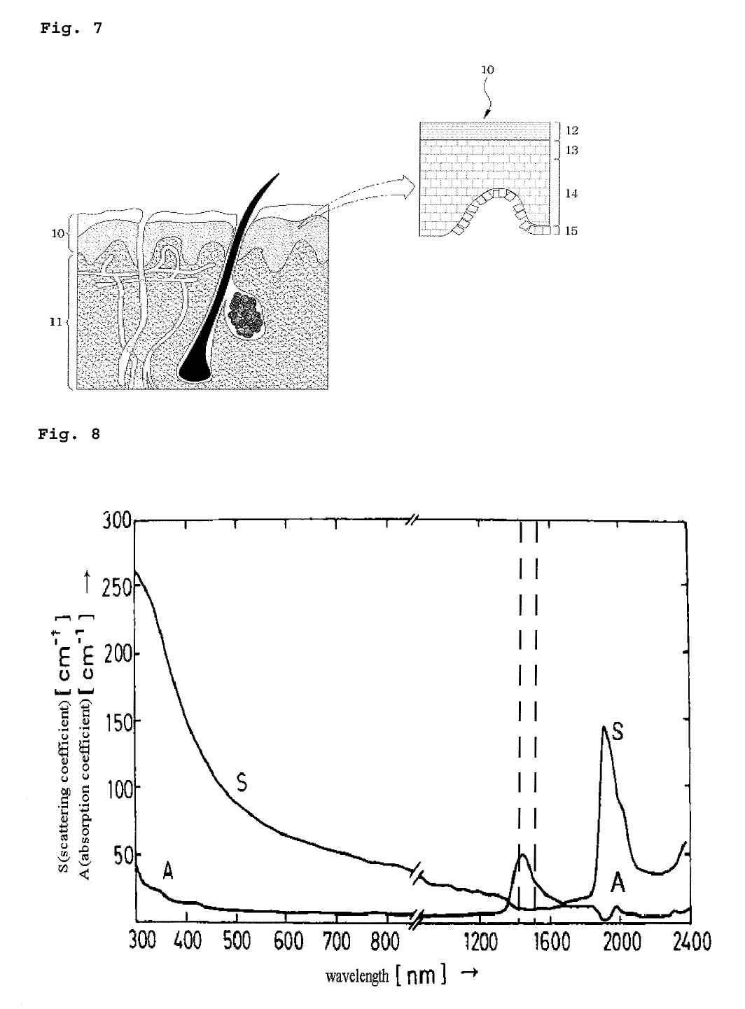

FIG. 7 is a cross-sectional view showing the structure of a general skin.

As shown in FIG. 7, the skin comprises an epidermis 10 and a dermis 11 under keratin.

The epidermis 10 consists of the following four layers under a sebaceous film: a horny layer (=stratum corneum) 12, a granular layer (=stratum granulosum) 13, a spinous layer (=stratum spinosum) 14 and a basal cell layer (=stratum basale) 15. A skin cell is produced in the basal cell layer.

The produced skin cell is moved toward an upper layer while changing its shape and then is gradually changed into a keratin. Ultimately, the keratin becomes the dirt which will in turn be removed from the skin. Likewise, the skin is continuously produced and changed.

The dermis 11 supports the basal cell layer 15 playing an important role in producing the skin cell. The dermis 11 is a key place which influences the moisture retention power of the epidermis 11, and maintains elasticity of the skin to thereby make the skin taut.

A large portion of the dermis 16 is occupied by collagen. The reason why the skin is aged, or chloasma, wrinkle, freckle and the like occur in a large amount on the skin is because collagen is insufficient or its quality is degraded.

In the meantime, the correlation between the skin and the optical energy is directly related to a wavelength of the optical energy as well as skin absorption and scattering of the optical energy.

Thus, the production of collagen in the dermis 11 must be first performed in order to medically treat scar and pigmentation of skin.

The wavelength the most suitable for the production of collagen is within a range between 1400 nm and 1500 nm as shown in FIG. 8. The reason for this is that an absorption coefficient (A) of the dermis layer is the highest and a scattering coefficient (B) of the dermis layer is the lowest in the above wavelength range.

Also, in case where the optical energy absorbed into the skin is converted into heat energy, if water is not absorbed into the skin, thermal damage is applied to the skin tissue surrounding the skin surface, leading to a reduction in a medical treatment effect.

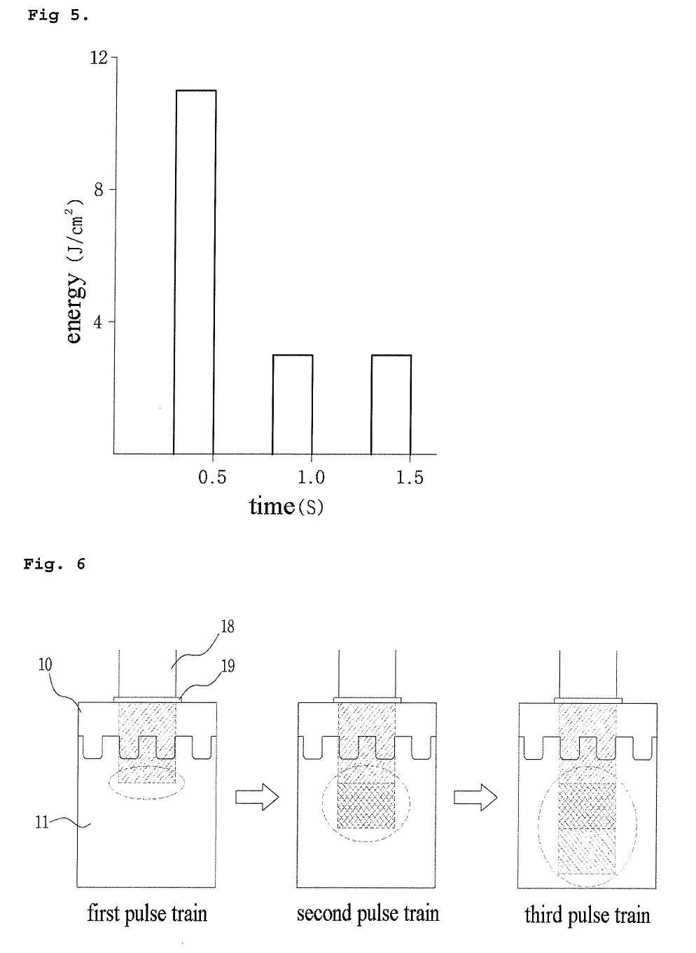

As shown in FIG. 5, since a high water absorption is exhibited in the wavelength ranging from 1400 nm from 1500 nm, heat can be transferred to only a to-be-treated region. The use of the wavelength of the above range enables the selective treatment of the skin.

Meanswhile, a degree at which the optical energy of the laser pulse damages a living tissue is associated with how fast the energy is absorbed so as to be converted into heat. If heat induced by the laser pulse is transferred to a to-be-treated region for a long time period, a secondary wave of the transferred heat can be moved to the surrounding tissue of the to-be-treated region, thereby causing a secondary thermal damage.

However, if a laser pulse is transferred to the to-be-treated region with a high peak power during a very short pulse period, heat produced from the tissue does not have sufficient time for the heat to be dispersed to the surrounding tissue and is transferred limitedly to only the to-be-treated region.

Thus, the duration of the laser pulse is limited so that thermal damage of the surrounding tissue associated with heat being conducted can be reduced. When it is assumed that the time required for 50% of heat produced from the tissue of a to-be-treated region to be transferred to the surrounding region is a thermal relaxation time (TRT), if a laser beam is transferred to the to-be-treated region within the time shorter than the thermal relaxation time of the tissue, the produced heat does not have sufficient time for the heat to be transferred to the surrounding tissue, resulting in a reduced heat damage of the surrounding tissue

Of course, although if a laser pulse is transferred to the to-be-treated region with a high peak power by selecting a very short pulse, a tissue of the to-be-treated region may be damaged and demoisturized limitedly while applying a minimum secondary thermal damage to a region surrounding the tissue of the to-be-treated region.

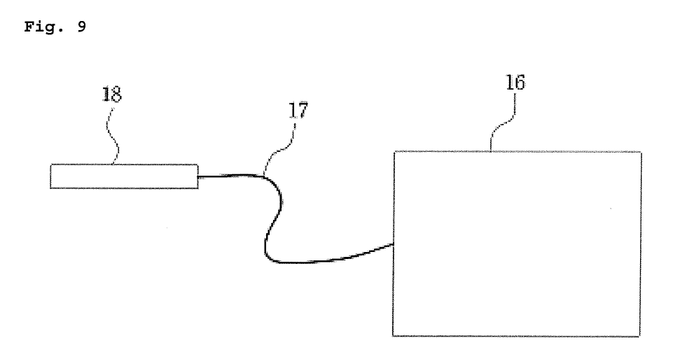

FIG. 9 is a diagrammatic view showing the construction of a laser apparatus for medical treatment using according to a preferred embodiment of the present invention.

Referring to FIG. 9, the laser apparatus for medical treatment using according to a preferred embodiment of the present invention includes a laser pulse generator 16 for generating a plurality of laser pulses each having a wavelength between 1400 and 1500 nm, an optical energy transfer element 17 connected at one end thereof to the laser pulse generator and for transferring optical energy of the laser pulses generated from the laser pulse generator, and a handpiece 18 connected at one end thereof to the other end of the optical energy transfer element and including a cooling unit 19 for cooling the skin of the to-be-treated region.

The laser pulse generator 16 sequentially generates one or more laser pulses having a wavelength between 1400 and 1500 nm. In this case, the pulse width of each of the laser pulses ranges from 50 ms to 300 ms, the interval between the laser pulses ranges from 50 ms to 300 ms, and the maximum power of the laser pulse is 40 W.

If the pulse width of each of the laser pulses is smaller than 50 ms, the dermis layer is less activated due to the transfer of low optical energy to the dermis layer. On the other hand, if the pulse width of each of the laser pulses is greater than 50 ms, the dermis rises in temperature due to the transfer of high optical energy to the dermis layer, thereby causing a damage to the epidermis and dermis layers.

In addition, if the interval between the laser pulses is smaller than 50 ms, high optical energy is transferred to the epidermis before the epidermis drops in temperature, thereby causing a damage to a to-be-treated region. On the other hand, if the interval between the laser pulses is smaller than 300 ms, although the energy is transferred to the to-be-treated region due to very low temperature of the epidermis, the effect of the pulse trains cannot be obtained.

Moreover, the diameter of a spot formed by each of the laser pulses irradiated to the to-be-treated region preferably ranges from 5 mm to 15 mm. The reason for this is because if the diameter of a spot formed by each of the laser pulses irradiated to the to-be-treated region is smaller than 5 mm, a treatment period is extended whereas if the diameter of the spot is greater than 15 mm, the optical energy of the laser pulse is well not transferred to the dermis layer of the to-be-treated region due to low energy density.

Like this, laser pulses are irradiated to a to-be-treated region with a high peak power during a short time period, such that although a tissue of the to-be-treated region is damaged and demoisturized limitedly while applying a minimum secondary thermal damage to a region surrounding the tissue of the to-be-treated region, a pain felt at the skin of the to-be-treated region remains as it is.

The sense of pain is sensation to pain felt in the skin. Any stimulus is so very strong that when a detrimental action is exerted on the human body, a pain is felt. The sensory receptors are dendritic free nerve terminals of the sensory nerves distributed widely in all the regions on the body surface except viscera in the body.

Each of the free nerve terminals is divided into the following two layers of the subcutis: a superficial part and a deep part. The free nerve terminal allows a person to distinguish a prickly pain and a sustaining pain. For example, when the skin surface is stimulated by a sharp distal end of a stiff hair, there are a point where pain is felt and a point where no pain is felt. The former is called a pain point.

Thus, in order to remove the residual pain caused by the laser pulses, the cooling unit 19 is mounted at the handpeice 18 and then the skin of a to-be-treated region is cooled to a temperature of 3° C. to 10° C. during a short time period prior to irradiation of the laser pulses to the to-be-treated region.

In this case, if the cooling temperature of the skin of the to-be-treated region is below 3° C., the to-be-treated region is demoisturized so that it can be damaged. On the other hand, if the cooling temperature of the skin is above 10° C., the skin temperature of the to-be-treated region rises, resulting in a reduction of a pain-relieving effect. Therefore, the skin of the to-be-treated region is most preferably cooled to a temperature ranging from 3° C. to 10° C.

The cooling unit is roughly divided into a contact type and non-contact type. A representative non-contact type cooling unit employs a skin cooling method using cool air. Cool air generated by a coolant (i.e., specific gas) is used or a simple cool air is used.

The contact type cooling unit employs a crystal having a low refraction index and high temperature conductivity like sapphire and fused silica, or an electrical cooling method such as TEC to cool the skin through the direct contact with the skin.

The cooling unit 19 according to an embodiment of the present invention is a unit which uses a combination of the above methods to effectively cool the skin of the to-be-treated region. The purpose of using the combined contact type cooling method and non-contact type cooling method is to complement an advantage and a disadvantage of the two cooling methods on a basis of the fact that in case of the non-contact type cooling method using cool air, the temperature of the epidermis can be effectively controlled but it takes much time to control the temperature of the dermis positioned below the epidermis whereas in case of the contact type cooling method using crystal, the surrounding region of a region being cooled is well controlled but a region far away from the region being cooled is not well controlled.

In addition, the pulse width of each of the laser pulses generated from the laser pulse generator, the interval between the lasers pulses, the wavelength and power of each laser pulse, and the cooling temperature of the cooling unit can be controlled by a controller mounted in a microprocessor which is typically well known in the prior art.

The action of the laser in the treatment process is based on a conversion of the optical energy into heat energy. Heat energy absorbed into the skin causes a local rise in temperature at a skin tissue irradiated with a laser beam.

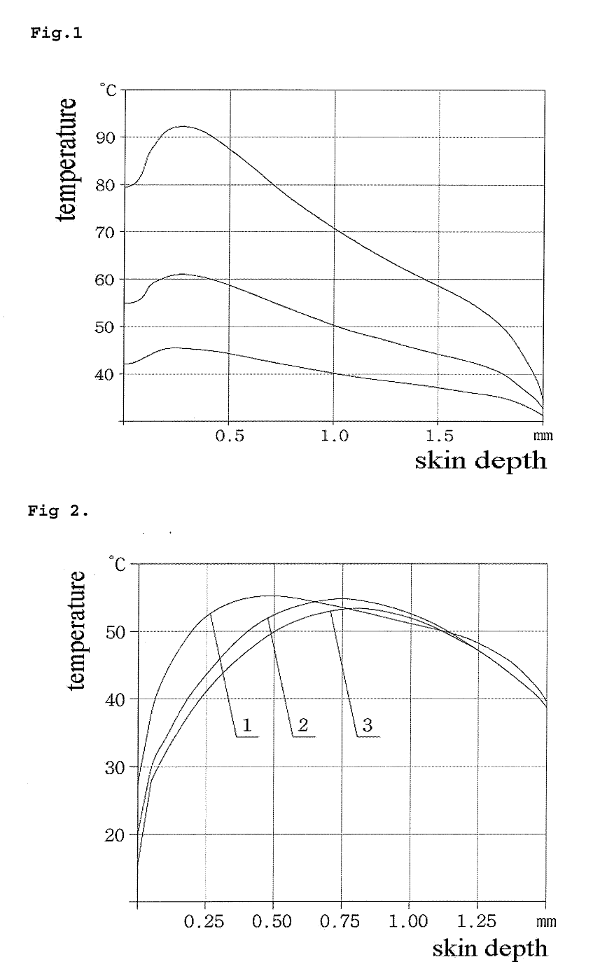

If the temperature is somewhat below a critical temperature for the phase transition from solid or liquid to gas, it rises in proportional to energy density (see FIG. 1). The thermal characteristics of a biologic tissue are determined depending on heat conductivity, heat emission of a vascular system, heat retention capacity of the tissue. The main effects observed in the tissue during the heating are shown in Table 1 below.

TABLE 1

| | | Biological | | |
|---|---|---|---|---|
| Temperature | Heating process | effect | Optical effect | Mechanical effect |
| Below 40° C. | Heated | No Permanent | Not observed | No |
| | | deformity | | |
| 40-60° C. | Heated | Enzyme, hydrops, | Slight | No |
| | | activation of | hyperemia of | |
| | | biologic | the tissue | |
| | | membrane, | | |
| | | variation in | | |
| | | latent ability | | |
| | | of membrane | | |
| | | (protein | | |
| | | denaturation) | | |
| 60-100° C. | Heated, | Skin tissue | Variation in | Decrease in tissue |
| | evaporation of | necrosis | color of the | density, steam rises |
| | received medium | possible | tissue, | |
| | | (coagulation of | increase in | |
| | | protein), | disperse | |
| | | dehydration of | | |
| | | the tissue | | |
| | | begins | | |
| Above 150° C. | Intensive | Carbonization of | The tissue | Considerable |
| | evaporation, | the tissue | assumes black | mechanical damage |
| | burned | | color and | |
| | | | causes | |
| | | | intensive | |
| | | | absorption. | |
| Above 300° C. | Combustion, | Complete | The optical | Removal of the tissue |
| | generation of | dehydration of | property is | by emission of gas and |
| | hydrogen from | the tissue | determined by | solid |
| | non-aqueous | | the shape of | particles-dispersion |
| | solution medium | | smoke and gas. | effect |

During the transfer of the heat, a part of the heat is emitted through the conducted heat and the vascular system of an adjoining tissue, and only partial residual heat is accumulated to thereby cause a heat variation in a region exerted with an effect of an optical beam.

### COMPARATIVE EXAMPLE

In a state where the skin is not cooled, the temperature variation of the skin according to the amount of energy upon the irradiation of the optical energy of the laser pulses to the skin of a to-be-treated region is shown in FIG. 1.

FIG. 1 is a graph showing a variation in temperature at the epidermis and the dermis when the optical energy of the laser pulses is irradiated to the skin for 200 ms without cooling the skin.

In FIG. 1, when the optical energy per unit area of 1 cm² is (1) 6.5 J/cm², (2) 13 J/cm² and (3) 26 J/cm², the temperature variations of the skin are shown respectively.

As shown in FIG. 1, it can be seen that the temperature of the skin decreases as the skin depth increases due to the heat conductivity and the vascular system.

In case where the skin is not cooled, as shown in FIG. 1, it can be seen that the temperature of the epidermis rises in proportion to the magnitude of the optical energy of the laser pulses being incident to the skin. Also, it can be seen from FIG. 1 that the epidermis does not maintain the temperature suitable for the production of collagen and its temperature drops sharply.

### TEST EXAMPLE (SIMULATION)

As shown in FIG. 1, it can be seen that the temperature of the skin decreases as the skin depth increases due to the heat conductivity and the vascular system.

This test was performed under the condition in which the skin penetration depth of the optical energy of laser pulses is 1.5 mm at the maximum, the temperature of the epidermis ranges from 50° C. to 55° C., and the wavelength of the incident laser pulse ranges from 1400 nm to 1500 nm.

The thermal and physical characteristics of the skin vary depending on layers constituting the skin, and the skin layers are composed of a complex structure. Thus, this test was conducted on the epidermis layer and the dermis layer except a horny layer.

The heat transfer coefficient and the volumetric heat capacity for each layer are shown in Table 2 below.

TABLE 2

| | heat transfer | volumetric heat capacity |
|---|---|---|
| | coefficient(λ) | (CO) |
| Epidermis (−0.1 mm) | 0.266 W/mK | 0.5 W/mK |
| Dermis (−1.9 mm) | 5.92 × 10⁶ J/mK | 3.2 × 10⁶ J/mK |

The model of the skin layers has been simulated since 1989, and a one-dimensional linear approximation was performed on a model including various boundary conditions in the present invention.

The boundary conditions are expressed by the following Equations 1 and 2:

$$\begin{matrix}\begin{matrix}\frac{1}{{a}_{1}} \frac{\partial {t}_{1}}{\partial τ} = \frac{{\partial }^{2} {t}_{1}}{\partial {x}^{2}} + \frac{{w}_{1} ( x )}{{λ}_{1}} ; & ( a ) \\ x = 0 \text{:} \frac{d {t}_{1}}{d x} = \frac{α}{{λ}_{1}} ( {t}_{1} - {t}_{c} ) ; & ( b ) \\ x = {L}_{1} \text{:} {λ}_{1} \frac{d {t}_{1}}{d x} = {λ}_{2} \frac{d {t}_{2}}{d x} ; & ( c ) \\ τ = {τ}_{0} \text{:} t = {t}_{0}^{1} ( x ) ; & ( d )\end{matrix} \} epidermis & [ Equation 1 ] \\ \begin{matrix}\frac{1}{{a}_{2}} \frac{\partial {t}_{2}}{\partial τ} = \frac{{\partial }^{2} {t}_{2}}{\partial {x}^{2}} + \frac{{w}_{2} ( x )}{{λ}_{2}} ; & ( a ) \\ x = {L}_{1} \text{:} {λ}_{2} \frac{d {t}_{2}}{d x} = {λ}_{1} \frac{d {t}_{1}}{d x} ; & ( b ) \\ x = {L}_{2} \text{:} t = {t}_{0} ; & ( c ) \\ τ = {τ}_{0} \text{:} t = {t}_{0}^{2} ( x ) ; & ( d )\end{matrix} \} Dermis & [ Equation 2 ]\end{matrix}$$

Here, a: heat capacity coefficient (m²/s) of cooling material
t: temperature of skin layer(° C.)
τ: time (sec)
α: thermal conductivity of the skin layer (W/m²K)
λ: heat capacity coefficient (W/mK) of the skin layer
l: thickness (m) of the skin layer
t₀: initial temperature (° C.) of the skin layer
t_{c}: temperature (° C.) of coolant

The present invention used one or more pulse trains so as to be incident on the skin after cooling the skin to 4° C. for 300 ms in order to suppress a temperature rise of the epidermis and maintain the temperature suitable for the activation of the dermis layer.

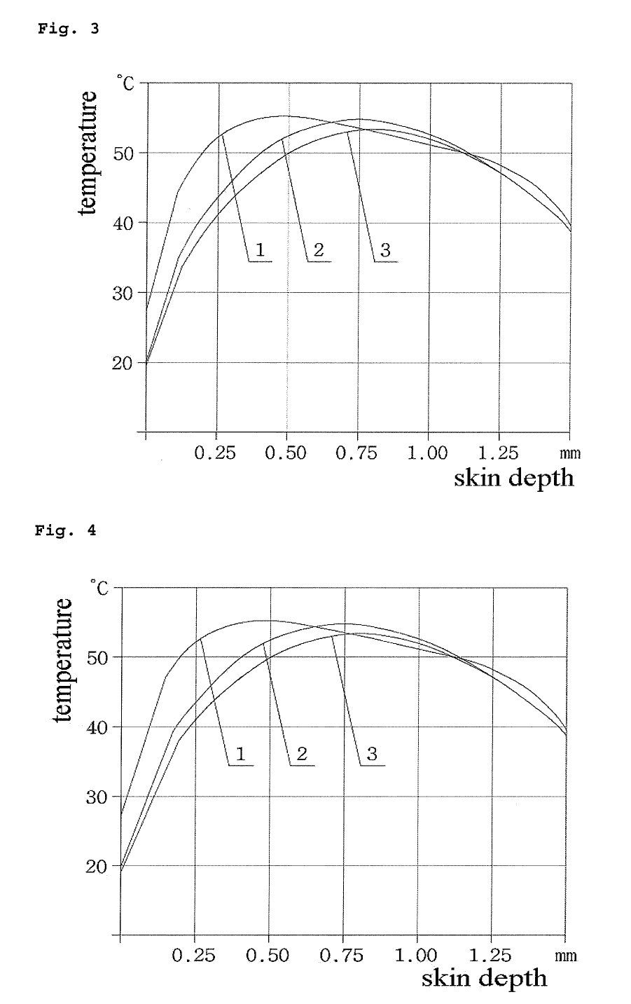

FIGS. 2 to 4 are graphs showing variations in temperature according to the skin depth at the epidermis when the trains of the laser pulses are incident on the epidermis with depths of 50 μm, 100 μm and 150 μm, respectively, after cooling the skin.

In FIGS. 2 to 4, the pulse width of each of the laser pulses is 200 ms, the interval between the laser pulses is to 300 ms, and the optical energy per unit area of 1 cm² is (1) 11 J/cm², (2) 3 J/cm² and (3) 3 J/cm².

FIG. 5 is a graph showing the relationship between the magnitude of optical energy transferred to the skin and the interval between laser pulses according to an embodiment of the present invention.

As shown in FIGS. 2 to 4, in case where the skin to 4° C. for 300 ms is cooled by using the cooling unit and then one or more pulse trains are incident on the skin, the dermis layer is activated widely and deeply by the pulse trains and the heat transferred to the epidermis is nearly negligible by the cooling of the skin. It could be found from the results of FIGS. 2 to 4 that the epidermis and the dermis are maintained at the temperature suitable for the protein denaturation while activating the dermis layer irrespective of the thickness of the epidermis.

FIG. 6 is a diagrammatic view showing a degree in which heat is dispersed in the skin by the pulse trains.

As shown in FIG. 6, it is possible to intuitionally grasp an activation degree of the dermis layer by first, second and third pulse trains.

As described above, a laser apparatus for medical treatment using pulse trains according to the present invention has the following advantages:

First, the laser apparatus of the present invention is relatively simple in structure, making it easy to manufacture and is relatively reduced in its manufacturing cost as compared to the conventional prior art.

Second, a pain caused in the conventional prior art can be remarkably lessened.

Third, the region surrounding a to-be-treated region can be minimally damaged to increase the frequency of treatments.

Fourth, maintenance and repair of the inventive laser apparatus are convenient due to its simple structure.

Last, sequelae caused after treatment are minimized.

The invention has been described in detain with reference to preferred embodiments thereof. However, it will be appreciated by those skilled in the art that changes may be made in these embodiments without departing from the principles and spirit of the invention, the scope of which is defined in the appended claims and their equivalents.

1. A laser apparatus for medical treatment using pulse trains, comprising:
a laser pulse generator for generating a plurality of laser pulses;
an optical energy transfer element connected at one end thereof to the laser pulse generator for transferring optical energy of the laser pulses generated from the laser pulse generator;
a handpiece connected at one end thereof to the other end of the optical energy transfer element for irradiating the optical energy of the laser pulses transferred thereto from the laser pulse generator by the optical energy transfer element to a to-be-treated region while being close contact with the to-be-treated region;
a cooling unit mounted at a distal end of the handpiece for cooling the skin of the to-be-treated region; and
a controller for adjusting the width of and the optical energy of the laser pulses generated from the laser pulse generator in response to an external input signal, and controlling a skin cooling temperature through the cooling unit,
whereby the skin of the to-be-treated region is cooled to a given temperature by the cooling unit, and then the plurality of laser pulses generated from the laser pulse generator are irradiated to the to-be-treated region at specific intervals.

2. The laser apparatus of claim 1, wherein the wavelength of each of the laser pulses generated from the laser pulse generator ranges from 1400 nm to 1500 nm.

3. The laser apparatus of claim 1, wherein the pulse width of each of the laser pulses generated from the laser pulse generator ranges from 50 ms to 300 ms.

4. The laser apparatus of claim 1, wherein the interval between the laser pulses generated from the laser pulse generator ranges from 50 ms to 300 ms.

5. The laser apparatus of claim 1, wherein the diameter of a spot formed by each of the laser pulses irradiated to the to-be-treated region through the handpiece ranges from 5 mm to 15 mm.

6. The laser apparatus of claim 1, wherein the cooling temperature of the skin cooled through the cooling unit ranges from 3° C. to 10° C.

7. The laser apparatus of claim 2, wherein the cooling temperature of the skin cooled through the cooling unit ranges from 3° C. to 10° C.

8. The laser apparatus of claim 3, wherein the cooling temperature of the skin cooled through the cooling unit ranges from 3° C. to 10° C.

9. The laser apparatus of claim 4, wherein the cooling temperature of the skin cooled through the cooling unit ranges from 3° C. to 10° C.

10. The laser apparatus of claim 5, wherein the cooling temperature of the skin cooled through the cooling unit ranges from 3° C. to 10° C.
